(19) United States
(12) Patent Application Publication
(10) Pub. No.: US 2025/0213399 A1
(43) Pub. Date: Jul. 3, 2025

(54) SOFT AND DRY DOUBLE-LAYER COMPOSITE MATERIAL, PREPARATION DEVICE AND PREPARATION METHOD THEREFOR AND HYGIENIC PRODUCT

(71) Applicant: KING'S CHOICE (JIANGSU) NEW MATERIAL CO., LTD., Jiangsu (CN)

(72) Inventors: Xueen HAO, Jiangsu (CN); Min ZHAI, Jiangsu (CN); Qiangqiang TAO, Jiangsu (CN)

(73) Assignee: KING'S CHOICE (JIANGSU) NEW MATERIAL CO., LTD., Jiangsu (CN)

(21) Appl. No.: 18/852,505

(22) PCT Filed: Nov. 10, 2022
(86) PCT No.: PCT/CN2022/131132

§ 371 Date: Dec. 5, 2024

(30) Foreign Application Priority Data
Mar. 31, 2022 (CN) 202210330668.7

(51) Int. Cl. A61F 13/512; A61F 13/15

(52) U.S. Cl. CPC A61F 13/5123; A61F 13/15731; A61F 13/15764; A61F 13/15804; A61F 2013/15016; A61F 2013/15121; A61F 2013/15146; A61F 2013/1517; A61F 2013/15463; A61F 2013/15512; A61F 2013/15552; A61F 2013/15991

(57) ABSTRACT

A soft and dry double-layer composite material includes a bottom layer and a surface layer arranged on the bottom layer. The surface layer has an upper surface and a lower surface. The surface layer is provided with openings penetrating the surface layer. The bottom layer is provided with first protrusions protruding upwards. The first protrusions penetrate the openings respectively. A top surface of each of the first protrusions is higher than the upper surface of the surface layer. The bottom layer is made of finer fibers than the surface layer.

### TECHNICAL FIELD

The present disclosure relates to the technical field of double-layer composite materials, and in particular, to a soft and dry double-layer composite material, a preparation device and a preparation method thereof, and a hygienic product.

### BACKGROUND

As daily necessities for people, absorbent hygienic products including sanitary napkins, diapers, pull-up pants, adult diapers, adult pull-up pants, mild incontinence products, breast pads, and absorbent pads bring great convenience to people's lives. Taking sanitary napkins as an example, the current market penetration rate of sanitary napkins in China is basically 100%, which means that every consumer experiencing menstruation already uses sanitary napkins. The market penetration rate of baby diapers and pull-up pants has basically reached about 80% this year and will continue to increase in the next few years.

In order to meet consumers' growing pursuit of comfort and softness, more and more fabrics are made of finer fibers. Meanwhile, in order to pursue dryness, weakly hydrophilic fibers are often used or weakly hydrophilic finishing is performed after the fabrics are made. The weakly hydrophilic treatment can reduce seepage caused by reverse infiltration of the liquid in the core through the fabric when the core is under pressure. However, it is difficult for these two trends to make the fabrics delicate, soft, and dry at the same time. The reasons are as follows: (1) Fine fibers can help improve softness and comfort, especially when the fabric is dry. However, as an absorbent product, the reduction in fineness will also greatly increase the number of fibers per unit area, reduce the gaps between fibers, and reduce the fluffiness of the fabric. When the fabric absorbs liquid, the small gaps between fibers slow down liquid infiltration and increase the storage of liquid in the fabric, which increases the liquid holding amount and liquid holding rate of the surface layer, increases the wetness of the fabric and the amount of seepage, and affects the dryness of the fabric and the consumer comfort. (2) The weakly hydrophilic treatment will lead to reduced hydrophilicity of fibers and fabrics, and the time for liquid to infiltrate the fabric will be prolonged. Meanwhile, a situation similar to the lotus effect will occur and the liquid cannot infiltrate, resulting in slippage and side leakage and affecting the function of the absorbent product and the consumer experience. The leakage problem also causes a lot of trouble for consumers, such as staining underwear or bed sheets.

A fabric made by simply mixing coarse denier fibers and fine denier fibers and adjusting the fibers by proportion can only improve dryness and comfort to a limited extent, but cannot significantly alleviate or resolve the contradiction between the two to achieve a balance.

Another trend is that more and more fabrics are made of natural fibers such as spunlace pure cotton or spunlace viscose fibers. However, these pure cotton materials featuring liquid absorption and retention cannot achieve good dryness. In view of the above, it is necessary to develop an innovative fabric to improve dryness.

### SUMMARY

To solve the deficiencies of the prior art, the present disclosure provides a soft and dry double-layer composite material, a preparation device and a preparation method thereof, and a hygienic product, to ensure that a fabric is dry, soft, and skin-friendly.

To achieve the above objective, the present disclosure adopts the following technical solutions.

A soft and dry double-layer composite material includes a bottom layer and a surface

layer arranged on the bottom layer, where the surface layer has an upper surface and a lower surface, and the surface layer is provided with openings penetrating the surface layer; the bottom layer is provided with first protrusions protruding upwards, the first protrusions penetrate the openings respectively, and a top surface of each of the first protrusions is higher than the upper surface of the surface layer; a material for the bottom layer has a fineness less than a material for the surface layer.

Preferably, the material for the bottom layer has weaker hydrophilicity than the material for the surface layer.

Preferably, the surface layer is provided with second protrusions, and the openings are located in top surfaces of the second protrusions respectively.

Preferably, a side wall of each of the first protrusions located in a corresponding one of the second protrusions is completely in contact with, partially in contact with, or not in contact with an inner wall of the corresponding one of the second protrusions to form a cavity.

Preferably, the surface layer is provided with second protrusions, the openings are located in the surface layer at positions other than the second protrusions, and a top surface of each of the second protrusions is lower than the top surface of each of the first protrusions.

Preferably, an upper surface of the bottom layer and the lower surface of the surface layer are at least partially bonded and fixed.

Preferably, each of the first protrusions is provided with a bottom layer hole penetrating the bottom layer.

Preferably, each of the second protrusions is provided with a surface layer hole penetrating the surface layer.

Preferably, first through-holes and second through-holes respectively penetrating the bottom layer and the surface layer are provided at contact parts between the bottom layer and the surface layer, the first through-holes are in communication with the second through-holes, and the first through-holes and the second through-holes are identical or different in size.

The present disclosure also provides a device for preparing the soft and dry double-layer composite material, which includes a first roller, a second roller, a concave roller, and a bonding mechanism, where
a plurality of first opening members for making the openings are provided on a roller surface of the first roller;
the concave roller cooperates with the first roller, and a plurality of second opening members matching with the first opening members are provided on a roller surface of the concave roller, and the second opening members cooperate with the first opening members to form, on the surface layer, the openings penetrating the surface layer;
the second roller cooperates with the concave roller, and a plurality of first bosses for pressing out the first protrusions on the bottom layer are provided on a roller surface of the second roller, a plurality of first grooves matching with the first bosses are provided in the roller surface of the concave roller, and the first grooves and the first bosses cooperate to form, on the bottom layer, the first protrusions penetrating the openings in the surface layer; and
the bonding mechanism is configured for bonding the contact parts between the upper surface of the bottom layer and the lower surface of the surface layer.

Preferably, a plurality of second bosses for pressing out the second protrusions on the surface layer are provided on the roller surface of the first roller, a plurality of second grooves matching with the second bosses are provided on the roller surface of the concave roller, and the second grooves and the second bosses cooperate to form the second protrusions on the surface layer.

The present disclosure also provides a method for preparing the soft and dry double-layer composite material. The method uses the device for preparing the soft and dry double-layer composite material.

The method includes the following steps:
S1. conveying the surface layer between the concave roller and the first roller, and forming the openings penetrating the surface layer through cooperation of the first opening members and the second opening members;
S2. conveying the surface layer between the concave roller and the second roller, and forming, on the bottom layer, the first protrusions penetrating the openings in the surface layer through cooperation of the first grooves and the first bosses; and
S3. continuously conveying the surface layer and the bottom layer forward and combining the surface layer and the bottom layer to form the soft and dry double-layer composite material.

The present disclosure provides another method for preparing the soft and dry double-layer composite material. The method uses the device for preparing the soft and dry double-layer composite material.

The method includes the following steps:
S1. conveying the surface layer between the concave roller and the first roller, forming the second protrusions on the surface layer through cooperation of the second grooves and the second bosses, and forming the openings penetrating the surface layer on the second protrusions through cooperation of the first opening members and the second opening members;
S2. conveying the surface layer between the concave roller and the second roller, and forming, on the bottom layer, the first protrusions penetrating the openings in the surface layer through cooperation of the first grooves and the first bosses; and
S3. continuously conveying the surface layer and the bottom layer forward and combining the surface layer and the bottom layer to form the soft and dry double-layer composite material.

The present disclosure also provides a hygienic product, which includes the soft and dry double-layer composite material.

Preferably, the hygienic product includes a sanitary napkin, a sanitary pad, menstrual pants, a mild incontinence product, a diaper, pull-up pants, a nursing pad, a maternity napkin, or a breast pad.

### Advantages

As for the double-layer composite material of the present disclosure, a material of coarse denier fibers and a material of fine denier fibers are combined and the material of coarse denier fibers is lower than the material of fine denier fibers. Therefore, the material of fine denier fibers contacts skin of users and provides a maximized soft and comfortable user experience. The material of coarse denier fibers does not contact skin of users, which solves the problem of lacking softness and skin-friendliness of the coarse denier fabric. Meanwhile, the coarse denier fabric also makes up for the lack of dryness in the material of fine denier fibers, thereby improving the dryness of the fabric while ensuring a soft and comfortable experience.

### BRIEF DESCRIPTION OF THE DRAWINGS

FIG. 1 is a schematic structural diagram of a double-layer composite material according to an embodiment of the present disclosure;

FIG. 2 is a sectional diagram along A-A in FIG. 1;

FIG. 3 is a schematic structural diagram of a double-layer composite material according

to another embodiment of the present disclosure;

FIG. 4 is a sectional diagram along B-B in FIG. 3;

FIG. 5 is a schematic structural diagram of a double-layer composite material according to another embodiment of the present disclosure;

FIG. 6 is a sectional diagram along C-C in FIG. 5;

FIG. 7 is a schematic structural diagram of a double-layer composite material according to another embodiment of the present disclosure;

FIG. 8 is a sectional diagram along D-D in FIG. 7;

FIG. 9 is a schematic structural diagram of a double-layer composite material according to another embodiment of the present disclosure;

FIG. 10 is a sectional diagram along E-E in FIG. 9;

FIG. 11 is a schematic structural diagram of a device for preparing the double-layer composite material according to an embodiment of the present disclosure;

FIG. 12 is a schematic structural diagram of a device for preparing the double-layer composite material according to another embodiment of the present disclosure;

FIG. 13 is a schematic structural diagram of a double-layer composite material according to another embodiment of the present disclosure;

FIG. 14 is a schematic structural diagram of a double-layer composite material according to another embodiment of the present disclosure; and

FIG. 15 is a schematic structural diagram of a double-layer composite material according to another embodiment of the present disclosure.

Reference numerals in the drawings: 1. bottom layer, 1.1. first protrusion, 1.2. bottom layer hole, 1.3. first through-hole, 2. surface layer, 2.1. opening, 2.2. second protrusion, 2.3. surface layer hole, 2.4. second through-hole, 3. first roller, 3.1. first opening member, 3.2. second boss, 4. second roller, 4.1. first boss, 5. concave roller, 5.1. second opening member, 5.2. first groove, 5.3. second groove, 6. bonding mechanism.

### DETAILED DESCRIPTION OF THE EMBODIMENTS

The present disclosure will be described in detail below with reference to the accompanying drawings and specific embodiments.

### Embodiment 1

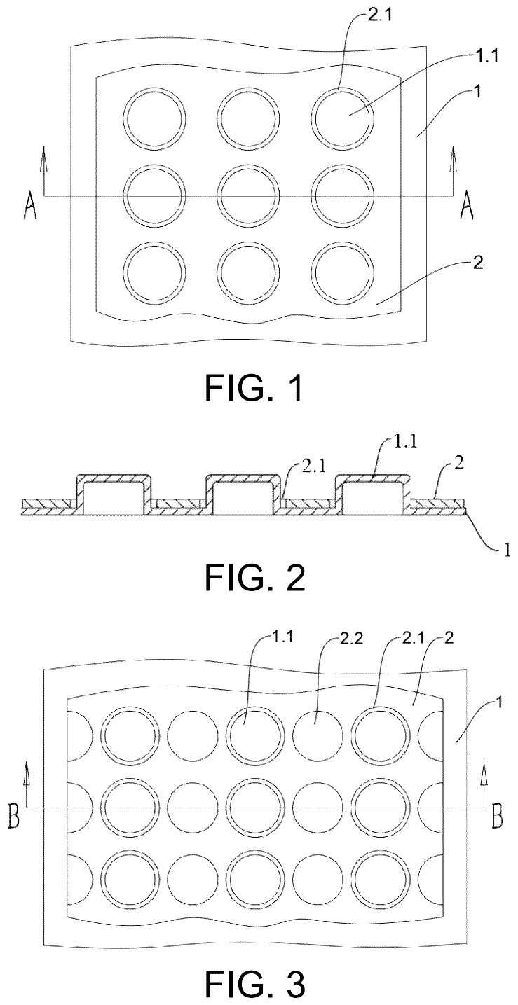

Referring to FIG. 1 and FIG. 2, a soft and dry double-layer composite material of the present disclosure includes a bottom layer 1 and a surface layer 2 arranged on the bottom layer 1. The surface layer 2 is provided with a plurality of openings 2.1 penetrating the surface layer 2. The bottom layer 1 is provided with a plurality of first protrusions 1.1 protruding upwards. The first protrusions 1.1 correspond to the openings 2.1, and the first protrusions 1.1 penetrate the openings 2.1 respectively. Contact parts between an upper surface of the bottom layer 1 and a lower surface of the surface layer 2 are bonded together. A top surface of each of the first protrusions 1.1 is higher than an upper surface of the surface layer 2. The bottom layer 1 is made of fine denier fibers, the surface layer 2 is made of coarse denier fibers, and the bottom layer 1 has weaker hydrophilicity than the surface layer 2.

In this structure, the surface layer 2 is mainly made of a non-woven fabric with coarser fibers, so that it shows better liquid permeability and air permeability and also has better liquid retention capacity, thereby effectively controlling seepage. The bottom layer 1 is made of a fabric with finer fibers and it protrudes through the openings in the surface layer 2 to directly contact human skin. The bottom layer 1 is skin-friendly, soft, and dry due to its fine fibers and weak hydrophilicity.

The surface layer 2 and the bottom layer 1 can be bonded between the non-opening area of the surface layer 2 and the non-protrusion area of the bottom layer 1, and the bonding part can be the entire contact area or a part of the contact area.

The bottom layer 1 is made of a hot-air non-woven fabric with 24-gsm 1.0-denier weakly hydrophilic fibers, and the surface layer 2 is made of a hot-air non-woven fabric with 20-gsm 2.5-denier strongly hydrophilic fibers. The first protrusions 1.1 on the bottom layer 1 are 1.2 mm high and each have a diameter of 6 mm. Each of the first protrusions 1.1 is provided with a bottom layer hole 1.2, and the diameter of the bottom layer hole 1.2 is 1 mm. The openings 2.1 in the surface layer 2 each have a diameter of 7 mm.

Preferably, both the surface layer 2 and the bottom layer 1 are made of hot-air non-woven fabrics, the upper surface of the bottom layer 1 is more delicate to touch and has finer fibers than the upper surface of the surface layer 2, the bottom layer 1 is made of pure cotton, the surface layer 2 is made of a bicomponent hot-air fabric, and the surface layer 2 and the bottom layer 1 are different in color, for example, the bottom layer 1 is white and the surface layer 2 is light green.

Preferably, the upper surface of the bottom layer 1 and the lower surface of the surface layer 2 are bonded by thermal bonding, adhesive bonding, or ultrasonic bonding.

Referring to FIG. 11, a device for preparing the soft and dry double-layer composite material is also provided in this embodiment. The device includes a first roller 3, a second roller 4, a concave roller 5, and a bonding mechanism 6. A plurality of first opening members 3.1 for making the openings 2.1 are provided on a roller surface of the first roller 3. The concave roller 5 cooperates with the first roller 3, and a plurality of second opening members 5.1 matching with the first opening members are provided on a roller surface of the concave roller 5. The second opening members 5.1 cooperate with the first opening members 3.1 to form the openings 2.1 penetrating the surface layer 2. The second roller 4 cooperates with the concave roller 5, and a plurality of first bosses 4.1 for pressing out the first protrusions 1.1 on the bottom layer 1 are provided on a roller surface of the second roller 4. A plurality of first grooves 5.2 matching with the first bosses 4.1 are provided in the roller surface of the concave roller 5. The first grooves 5.2 and the first bosses 4.1 cooperate to form, on the bottom layer 1, the first protrusions 1.1 penetrating the openings 2.1 in the surface layer 2. The bonding mechanism 6 is configured for bonding the contact parts between the upper surface of the bottom layer 1 and the lower surface of the surface layer 2.

In this embodiment, a method for preparing the soft and dry double-layer composite material includes the following steps.
S1. The surface layer 2 is conveyed between the concave roller 5 and the first roller 3, the openings 2.1 penetrating the surface layer 2 are formed through cooperation of the first opening members 3.1 and the second opening members 5.1, and an adhesive is applied on the lower surface of the surface layer 2 in contact with the upper surface of the bottom layer 1.
S2. The surface layer 2 is conveyed between the concave roller 5 and the second roller 4, and the first protrusions 1.1 penetrating the openings 2.1 in the surface layer 2 are formed on the bottom layer 1 through cooperation of the first grooves 5.2 and the first bosses 4.1.
S3. The surface layer 2 and the bottom layer 1 are continuously conveyed forward and are combined to form a double-layer composite material.

### Embodiment 2

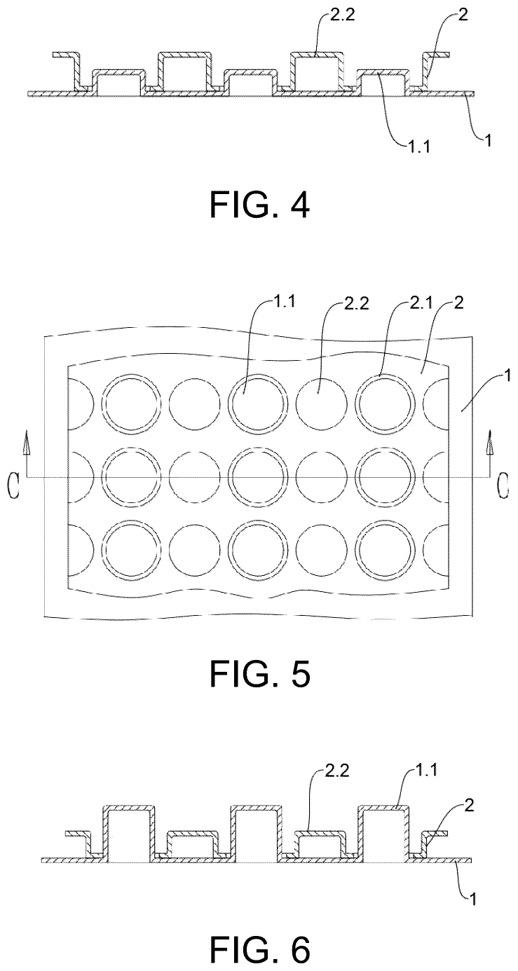

Referring to FIG. 3 and FIG. 4, a soft and dry double-layer composite material of the present disclosure includes a bottom layer 1 and a surface layer 2 arranged on the bottom layer 1. The surface layer 2 is provided with a plurality of second protrusions 2.2 protruding upwards. The surface layer 2 is provided with a plurality of openings 2.1 penetrating the surface layer 2. The bottom layer 1 is provided with a plurality of first protrusions 1.1 protruding upwards. The first protrusions 1.1 correspond to the openings 2.1, and the first protrusions 1.1 penetrate the openings 2.1 respectively. An outer side of each of the openings 2.1 is bonded to the lower surface of the surface layer 2.

The top surface of each of the second protrusions 2.2 is higher than the top surface of each of the first protrusions 1.1, so that the second protrusions 2.2 directly contact human skin. The surface layer 2 is made of a non-woven fabric with fine denier and weakly hydrophilic fibers. The bottom layer 1 is made of a coarse denier fabric. When the second protrusions 2.2 on the surface layer 2 contact human skin, a dry and soft effect is achieved. The first protrusions 1.1 formed on the bottom layer 1 have better anti-seepage and liquid retention capacities. The raised cavities improve the overall liquid retention capacity and the fluffiness of the fabric.

The surface layer 2 is made of a 23-gsm non-woven fabric capable of fading menstrual blood, the bottom layer 1 is made of a 20-gsm hot-air multi-time hydrophilic non-woven fabric, and the openings 2.1 each have a diameter of 7 mm.

Preferably, the top surface of each of the first protrusions 1.1 is lower than the top surface of each of the second protrusions 2.2, the surface layer 2 is made of fine denier fibers, the bottom layer 1 is made of coarse denier fibers, and the surface layer 2 has weaker hydrophilicity than the bottom layer 1. The surface layer 2 is made of a hot-air non-woven fabric with 22-gsm 0.8-denier weakly hydrophilic antibacterial fibers, and the bottom layer 1 is made of a hot-air non-woven fabric with 22-gsm 3.0-denier light green strongly hydrophilic fibers. The second protrusions 2.2 on the surface layer 2 are 1.5 mm high and each have a diameter of 6 mm. The first protrusions 1.1 on the bottom layer 1 are 0.8 mm high. The details can be seen in FIG. 3 and FIG. 4.

Referring to FIG. 5 and FIG. 6, the top surface of each of the first protrusions 1.1 is higher than the top surface of each of the second protrusions 2.2, the fabrics of the bottom layer 1 and the surface layer 2 are opposite to those in FIG. 3 and FIG. 4, the bottom layer 1 is made of fine denier fibers, the surface layer 2 is made of coarse denier fibers, and the bottom layer 1 has weaker hydrophilicity than the surface layer 2. The bottom layer 1 is made of a pure cotton spunlace non-woven fabric with 30-gsm pure cotton fibers, and the surface layer 2 is made of a hot-air non-woven fabric with 22-gsm 2.0-denier fibers. The first protrusions 1.1 on the bottom layer 1 are 1.3 mm high and each have a diameter of 6 mm. The second protrusions 2.2 on the surface layer 2 are 0.6 mm high and each have a diameter of 6 mm.

Preferably, the shapes and sizes of the openings 2.1, the first protrusions 1.1, and the second protrusions 2.2 can be changed as required.

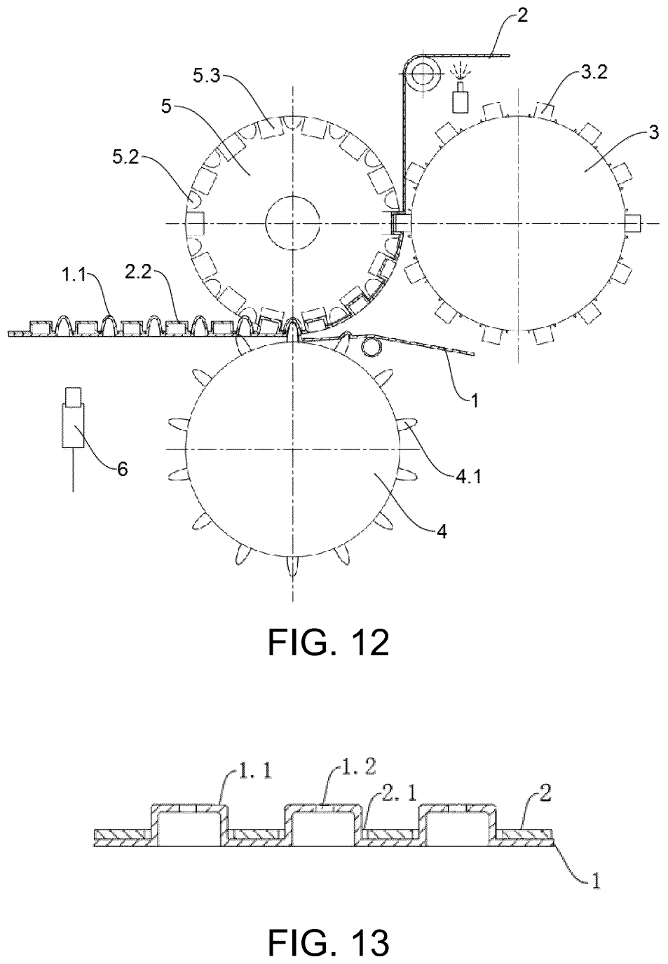

Referring to FIG. 12, a device for preparing the soft and dry double-layer composite material is also provided in this embodiment. The device includes a first roller 3, a second roller 4, a concave roller 5, and a bonding mechanism 6. A plurality of first opening members 3.1 for making the openings 2.1 are provided on a roller surface of the first roller 3. The concave roller 5 cooperates with the first roller 3, and a plurality of second opening members 5.1 matching with the first opening members are provided on a roller surface of the concave roller 5. The second opening members 5.1 cooperate with the first opening members 3.1 to form the openings 2.1 penetrating the surface layer 2. The second roller 4 cooperates with the concave roller 5, and a plurality of first bosses 4.1 for pressing out the first protrusions 1.1 on the bottom layer 1 are provided on a roller surface of the second roller 4. A plurality of first grooves 5.2 matching with the first bosses 4.1 are provided in the roller surface of the concave roller 5. The first grooves 5.2 and the first bosses 4.1 cooperate to form, on the bottom layer 1, the first protrusions 1.1 penetrating the openings 2.1 in the surface layer 2. The bonding mechanism 6 is configured for bonding the contact parts between the upper surface of the bottom layer 1 and the lower surface of the surface layer 2. A plurality of second bosses 3.2 for pressing out the second protrusions 2.2 on the surface layer 2 are arranged on the roller surface of the first roller 3. A plurality of second grooves 5.3 matching with the second bosses 3.2 are provided in the roller surface of the concave roller 5. The second grooves 5.3 and the second bosses 3.2 cooperate to form the second protrusions 2.2 on the surface layer 2.

In this embodiment, a method for preparing the soft and dry double-layer composite material includes the following steps.
S1. The surface layer 2 is conveyed between the concave roller 5 and the first roller 3, the second protrusions 2.2 are formed on the surface layer 2 through cooperation of the second grooves 5.3 and the second bosses 3.2, and the openings 2.1 penetrating the surface layer 2 are formed in the recessed portions through cooperation of the first opening members 3.1 and the second opening members 5.1.
S2. The surface layer 2 is conveyed between the concave roller 5 and the second roller 4, and the first protrusions 1.1 penetrating the openings 2.1 in the surface layer 2 are formed on the bottom layer 1 through cooperation of the first grooves 5.2 and the first bosses 4.1.
S3. The surface layer 2 and the bottom layer 1 are continuously conveyed forward and are combined to form a double-layer composite material.

### Embodiment 3

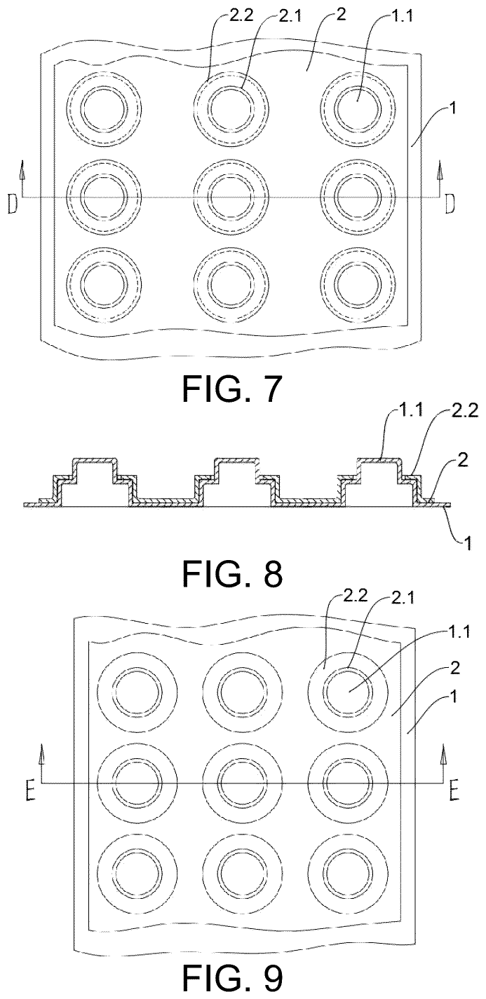

Referring to FIG. 7 and FIG. 8, a soft and dry double-layer composite material of the present disclosure includes a bottom layer 1 and a surface layer 2 arranged on the bottom layer 1. The surface layer 2 is provided with a plurality of second protrusions 2.2 protruding upwards. The second protrusions 2.2 are each provided with an opening 2.1 penetrating the second protrusion 2.2 on the surface layer 2. The bottom layer 1 is provided with a plurality of first protrusions 1.1 protruding upwards. The first protrusions 1.1 correspond to the openings 2.1, and the first protrusions 1.1 penetrate the openings 2.1 respectively. An outer side of each of the openings 2.1 is bonded to the lower surface of the surface layer 2. The top surface of each of the first protrusions 1.1 is higher than the upper surface of the surface layer 2. The bottom layer 1 is made of fine denier fibers, the surface layer 2 is made of coarse denier fibers, and the bottom layer 1 has weaker hydrophilicity than the surface layer 2.

Preferably, the side wall of the first protrusion 1.1 is bent, and at least a part of the side wall is in contact with the inner wall of the second protrusion 2.2. In this way, the firmness of contact with the side wall can be increased to improve the overall 3D retention effect of the fabric. More preferably, the side wall of the first protrusion 1.1 located inside the second protrusion 2.2 is completely in contact with and bonded to the inner wall of the second protrusion 2.2.

Preferably, the lower surface on the outer side of the opening 2.1 in the second protrusion 2.2 matches with and is bonded to the upper surface of the lower part of the first protrusion 1.1. The vertical section of the first protrusion 1.1 shows that the length of the upper part is smaller than the length of the lower part. The details can be seen in FIG. 7 and FIG. 8.

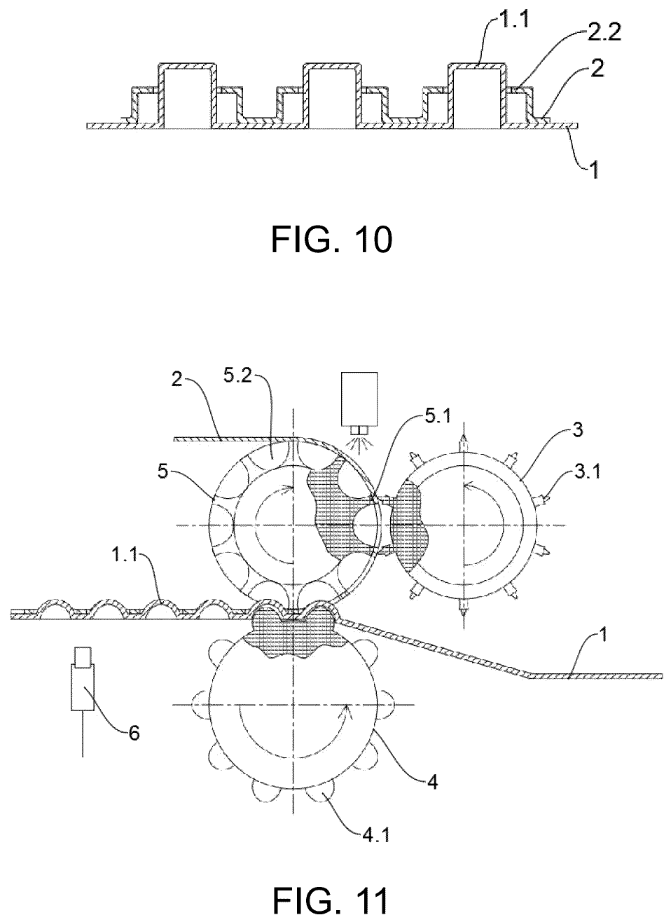

Referring to FIG. 9 and FIG. 10, the side wall of the first protrusion 1.1 is not bent, and a cavity is formed between the side wall of the first protrusion 1.1 and the inner wall of the second protrusion 2.2. This structure can be adopted to improve the overall fluffiness and liquid retention and anti-seepage capacities of the fabric through the formed cavities. Preferably, the side wall of the first protrusion 1.1 is a vertical surface perpendicular to the upper surface of the bottom layer 1.

A device for preparing the soft and dry double-layer composite material is also provided in this embodiment. The device includes a first roller 3, a second roller 4, a concave roller 5, and a bonding mechanism 6. A plurality of first opening members 3.1 for making the openings 2.1 are provided on a roller surface of the first roller 3. The concave roller 5 cooperates with the first roller 3, and a plurality of second opening members 5.1 matching with the first opening members 3.1 are provided on a roller surface of the concave roller 5. The second opening members 5.1 cooperate with the first opening members 3.1 to form the openings 2.1 penetrating the surface layer 2. The second roller 4 cooperates with the concave roller 5, and a plurality of first bosses 4.1 for pressing out the first protrusions 1.1 on the bottom layer 1 are provided on a roller surface of the second roller 4. A plurality of first grooves 5.2 matching with the first bosses 4.1 are provided in the roller surface of the concave roller 5. The first grooves 5.2 and the first bosses 4.1 cooperate to form, on the bottom layer 1, the first protrusions 1.1 penetrating the openings 2.1 in the surface layer 2. The bonding mechanism 6 is configured for bonding the contact parts between the upper surface of the bottom layer 1 and the lower surface of the surface layer 2. A plurality of second bosses 3.2 for pressing out the second protrusions 2.2 on the surface layer 2 are provided on the roller surface of the first roller 3. A plurality of second grooves 5.3 matching with the second bosses 3.2 are provided in the roller surface of the concave roller 5. The second grooves 5.3 and the second bosses 3.2 cooperate to form the second protrusions 2.2 on the surface layer 2.

In this embodiment, a method for preparing the soft and dry double-layer composite material includes the following steps.
S1. The surface layer 2 is conveyed between the concave roller 5 and the first roller 3, the second protrusions 2.2 are formed on the surface layer 2 through cooperation of the second grooves 5.3 and the second bosses 3.2, and the openings 2.1 penetrating the surface layer 2 are formed in the second protrusions 2.2 through cooperation of the first opening members 3.1 and the second opening members 5.1.
S2. The surface layer 2 is conveyed between the concave roller 5 and the second roller 4, and the first protrusions 1.1 penetrating the openings 2.1 in the surface layer 2 are formed on the bottom layer 1 through cooperation of the first grooves 5.2 and the first bosses 4.1.
S3. The surface layer 2 and the bottom layer 1 are continuously conveyed forward and are combined to form a double-layer composite material.

### Embodiment 4

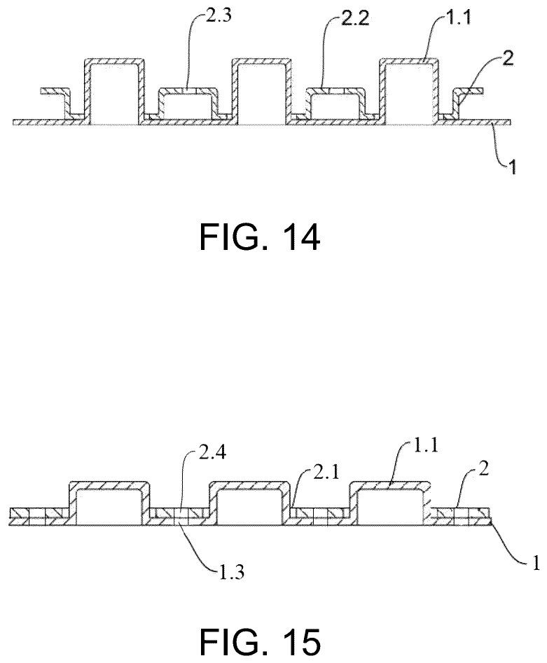

In order to improve the liquid permeability and air permeability, small holes can be provided in the bottom layer and the surface layer of the double-layer composite fabric of this patent. As shown in FIG. 13, bottom layer holes 1.2 are provided in the first protrusions 1.1 respectively. Holes can also be provided in the surface layer, and as shown in FIG. 14, surface layer holes 2.3 are provided in the second protrusions 2.2 respectively. Through-holes can also be provided in the bottom layer 1 and the surface layer 2, and as shown in FIG. 15, first through-holes 1.3 and second through-holes 2.4 respectively penetrating the bottom layer 1 and the surface layer 2 are provided at the contact parts between the bottom layer 1 and the surface layer 2. The first through-holes 1.3 are in communication with the second through-holes 2.4, and the first through-holes 1.3 and the second through-holes 2.4 are identical or different in size.

The basic principles, main features, and advantages of the present disclosure have been shown and described above. Those skilled in the art should understand that the above embodiments do not limit the present disclosure in any form, and any technical solution obtained by equivalent replacement or equivalent transformation falls within the protection scope of the present disclosure.

1. A soft and dry double-layer composite material, comprising a bottom layer and a surface layer arranged on the bottom layer, wherein
the surface layer has an upper surface and a lower surface, and the surface layer is provided with openings penetrating the surface layer;
the bottom layer is provided with first protrusions protruding upwards, the first protrusions penetrate the openings respectively, and a top surface of each of the first protrusions is higher than the upper surface of the surface layer; and
a material for the bottom layer has a fineness less than a material for the surface layer.

2. The soft and dry double-layer composite material according to claim 1, wherein the material for the bottom layer has weaker hydrophilicity than the material for the surface layer.

3. The soft and dry double-layer composite material according to claim 1, wherein the surface layer is provided with second protrusions, and the openings are located in top surfaces of the second protrusions respectively.

4. The soft and dry double-layer composite material according to claim 2, wherein a side wall of each of the first protrusions located in a corresponding one of the second protrusions is completely in contact with, partially in contact with, or not in contact with an inner wall of the corresponding one of the second protrusions to form a cavity.

5. The soft and dry double-layer composite material according to claim 1, wherein the surface layer is provided with second protrusions, the openings are located in the surface layer at positions other than the second protrusions, and a top surface of each of the second protrusions is lower than the top surface of each of the first protrusions.

6. The soft and dry double-layer composite material according to claim 1, wherein an upper surface of the bottom layer and the lower surface of the surface layer are at least partially bonded and fixed.

7. The soft and dry double-layer composite material according to claim 1, wherein each of the first protrusions is provided with a bottom layer hole penetrating the bottom layer.

8. The soft and dry double-layer composite material according to claim 3, wherein each of the second protrusions is provided with a surface layer hole penetrating the surface layer.

9. The soft and dry double-layer composite material according to claim 1, wherein first through-holes and second through-holes respectively penetrating the bottom layer and the surface layer are provided at contact parts between the bottom layer and the surface layer, the first through-holes are in communication with the second through-holes, and the first through-holes and the second through-holes are identical or different in size.

10. A device for preparing the soft and dry double-layer composite material according to claim 1, comprising a first roller, a second roller, a concave roller, and a bonding mechanism, wherein
a plurality of first opening members for making the openings are provided on a roller surface of the first roller;
the concave roller cooperates with the first roller, and a plurality of second opening members matching with the first opening members are provided on a roller surface of the concave roller, and the second opening members cooperate with the first opening members to form, on the surface layer, the openings penetrating the surface layer;
the second roller cooperates with the concave roller, and a plurality of first bosses for pressing out the first protrusions on the bottom layer are provided on a roller surface of the second roller, a plurality of first grooves matching with the first bosses are provided in the roller surface of the concave roller, and the first grooves and the first bosses cooperate to form, on the bottom layer, the first protrusions penetrating the openings in the surface layer; and
the bonding mechanism is configured for bonding the contact parts between the upper surface of the bottom layer and the lower surface of the surface layer.

11. The device for preparing the soft and dry double-layer composite material according to claim 10, wherein a plurality of second bosses for pressing out the second protrusions on the surface layer are provided on the roller surface of the first roller, a plurality of second grooves matching with the second bosses are provided on the roller surface of the concave roller, and the second grooves and the second bosses cooperate to form the second protrusions on the surface layer.

12. A method for preparing the soft and dry double-layer composite material, wherein the method uses the device for preparing the soft and dry double-layer composite material according to claim 10, and the method comprises the following steps:
S1. conveying the surface layer between the concave roller and the first roller, and forming, on the surface layer, the openings penetrating the surface layer through cooperation of the first opening members and the second opening members;
S2. conveying the surface layer between the concave roller and the second roller, and forming, on the bottom layer, the first protrusions penetrating the openings in the surface layer through cooperation of the first grooves and the first bosses; and
S3. continuously conveying the surface layer and the bottom layer forward and combining the surface layer and the bottom layer to form the soft and dry double-layer composite material.

13. A method for preparing the soft and dry double-layer composite material, characterized in that wherein the method uses the device for preparing the soft and dry double-layer composite material according to claim 11, and the method comprises the following steps:
S1. conveying the surface layer between the concave roller and the first roller, forming the second protrusions on the surface layer through cooperation of the second grooves and the second bosses, and simultaneously forming, on the second protrusions, the openings penetrating the surface layer through cooperation of the first opening members and the second opening members;
S2. conveying the surface layer between the concave roller and the second roller, and forming, on the bottom layer, the first protrusions penetrating the openings in the surface layer through cooperation of the first grooves and the first bosses; and
S3. continuously conveying the surface layer and the bottom layer forward and combining the surface layer and the bottom layer to form the soft and dry double-layer composite material.

14. A hygienic product, comprising the soft and dry double-layer composite material according to claim 1.

15. The hygienic product according to claim 14, wherein the hygienic product comprises a sanitary napkin, a sanitary pad, menstrual pants, a mild incontinence product, a diaper, pull-up pants, a nursing pad, a maternity napkin, or a breast pad.
